(19) United States
(12) Patent Application Publication
(10) Pub. No.: US 2003/0153590 A1
(43) Pub. Date: Aug. 14, 2003

(54) Method of treating alcoholism or alcohol abuse

(75) Inventors: Kauko O.A. Kurkela, Espoo (FI); Olli Puhakka, Littoinen (FI); Tuuli I. Sonck, Turku (FI); Sakari Karhuvaara, Turku (FI)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: OY CONTRAL PHARMA LTD

(21) Appl. No.: 10/217,151

(22) Filed: Aug. 13, 2002

Related U.S. Application Data
(60) Provisional application No. 60/330,510, filed on Oct. 23, 2001.
(60) Provisional application No. 60/311,796, filed on Aug. 14, 2001.

(51) Int. Cl. A61K 31/485

(52) U.S. Cl. 514/282

(57) ABSTRACT

The present invention relates to a method of treating alcoholism or alcohol abuse by administering to a subject a pharmaceutically effective amount of an opioid antagonist before imminent drinking. Particularly, the present invention relates to a method of treating alcoholism or alcohol abuse by administering transmucosally to a subject a pharmaceutically effective amount of an opioid antagonist before imminent drinking. Preferably, the opioid antagonist used in the method is nalmefene or a pharmaceutically acceptable salt thereof. The invention also relates to a method of treating alcoholism or alcohol abuse by administering to a subject before imminent drinking a transmucosal preparation comprising a pharmaceutically effective amount of an opioid antagonist, wherein the transmucosal preparation has rapid onset of action. Advantageously, a FAH+ subject is treated. Further, the invention relates to a method of treating alcoholism or alcohol abuse of a FAH+ subject, comprising extinguishing an alcohol-drinking response by administering to the FAH+ subject a pharmaceutically effective amount of nalmefene or a pharmaceutically acceptable salt thereof.

### BACKGROUND OF THE INVENTION

This application claims the priority benefit under 35 U.S.C. §119(e) of U.S. Provisional Application No. 60/311,796, filed Aug. 14, 2001, and U.S. Provisional Application No. 60/330,510, filed Oct. 23, 2001. The entirety of each of these documents is incorporated by reference herein.

### FIELD OF THE INVENTION

The present invention relates to a method of treating alcoholism or alcohol abuse. In one aspect, the present invention relates to a method of treating alcoholism or alcohol abuse in a family history positive (FAH+ or FHP) subject by administering to the FAH+ subject a pharmaceutically effective amount of an opioid antagonist, especially nalmefene.

In another aspect, the present invention relates to a method of treating alcoholism or alcohol abuse by administering to a subject a pharmaceutically effective amount of an opioid antagonist before imminent drinking. Specifically, a FAH+ subject is treated. Particularly, the present invention reelates to a method of treating alcoholism or alcohol abuse by administering transmucosally to a subject a pharmaceutically effective amount of an opioid antagonist before imminent drinking. More particularly, the invention relates to a method of treating alcoholism or alcohol abuse by administering to a subject a pharmaceutically effective amount of nalmefene or a pharmaceutically acceptable salt thereof transmucosally before imminent drinking. The invention also relates to a method of treating alcoholism or alcohol abuse by administering to a subject a transmucosal preparation comprising a pharmaceutically effective amount of an opioid antagonist before imminent drinking, wherein the transmucosal preparation has rapid onset of action.

### RELATED ART

Drinking alcohol (ethanol or ethyl alcohol) is a learned response, reinforced largely by the rewarding effects of alcohol in the central nervous system, the euphoria from lower, stimulatory doses of ethanol. An alcoholic is a person who, through an interplay of genetic and environmental factors, has had the alcohol-drinking response reinforced so often and so well that it becomes too strong for the individual to continue functioning properly in society. The strong alcohol-drinking response, i.e., the drive for alcohol, then dominates the person's behavior and life.

Alcoholism is the most costly health problem in many countries. Several treatment methods have been developed. According to Kranzler, despite the developments in treating alcoholism, such basic issues as the optimum dosing strategy and duration of treatment for existing therapies are not known (Kranzler, Alcohol & Alcoholism 35:537-547 (2000)). Some methods, such as counseling and Alcoholics Anonymous (AA), are aimed at increasing the alcoholic's ability or willpower to withstand the drive for alcohol. The drive, however, is not weakened and the patient is told that he will remain an alcoholic, that is, a person with an overly strong alcohol-drinking response, for the rest of his life. These methods succeed in some alcoholics, but in most cases eventually comes the time when a momentary decrease in willpower causes a resumption of alcohol drinking and alcohol abuse. These methods are not very successful because they do not effectively weaken the alcoholic's alcohol-drinking response.

Other treatments use punishment of various sorts (e.g., electric shock, disulfiram reactions, loss of a job) to try to stop alcohol drinking. Punishment is, however, a poor method for changing behavior and has many limitations. In particular, it is ineffective when positive reinforcement is still being received for the same response that is punished. Since the treatments that punish alcohol drinking do not block the positive reinforcement of the same response coming from alcohol in the brain, they should not be expected to be very effective.

In the most recent method, the alcohol-drinking response is extinguished by administering an opioid antagonist in conjunction with alcohol. Extinction consists of having the response emitted repeatedly in the absence of positive reinforcement. Much of the positive reinforcement for alcohol drinking is internal, from the rewarding effects of alcohol in the brain. U.S. Pat. No. 4,882,335 discloses a method for treating alcoholism in which the learned response of alcohol drinking is extinguished by being emitted while the reinforcement from alcohol in the brain is blocked with an opiate antagonist. In this extinction method, an opiate antagonist is administered to a subject suffering from alcoholism in a daily dosage sufficient to block the stimulatory effect of alcohol and, while the amount of antagonist in the subject's body is sufficient to block the stimulatory effect of alcohol, the subject is made to drink an alcoholic beverage. The steps of administering an opiate antagonist and drinking an alcoholic beverage are continued until the alcohol-drinking response is extinguished.

U.S. Pat. No. 5,086,058 describes the use of the opiate antagonist nalmefene in the above disclosed method for treating alcoholism. U.S. Pat. No. 5,086,058 describes that nalmefene can be given by injection, transdermal administration, nasal administration, suppository, sublingual administration, and the like, but oral administration is preferred. The daily dose of nalmefene is described as from 0.1 to 300 mg daily.

U.S. Pat. No. 5,096,715 describes a method for treating alcoholism by extinguishing the alcohol-drinking response by administering an opiate antagonist to a subject transdermally. U.S. Pat No. 5,096,715 describes the preferred opiate antagonists as naloxone and naltrexone, and the preferred dose ranges for naloxone and naltrexone as 0.2 to 40 mg daily and 20 to 300 mg daily, respectively.

Sinclair et al. describe the results of an open-label Finnish clinical trial of oral naltrexone in alcoholism treatment (Alcoholism Clin. Exp. Res. 25:127S-131S (2001); and Sinclair, Alcohol & Alcoholism 36:2-10 (2001)). The patients were instructed to take naltrexone orally one hour before drinking alcohol. Also, Heinala et al report clinical trials of oral naltrexone in alcoholism treatment (J. Clin. Psychopharmacol. 21:287-292 (2001)).

Oral nalmefene has been shown to be safe and effective for alcohol dependence (Mason et al., Alcohol Clin. Exp. Res. 18:1162-1167 (1994)). Sublingual naloxone has been tested for pain management (Weinberg et al., Clin. Pharmacol. Ther. 44:335-342 (1988)).

Typically, a person addicted to alcohol has a continuous craving (i.e., drive) for alcohol, and he or she is not able to anticipate when he or she will start drinking alcoholic beverages. Therefore, a treatment for alcoholism by extinguishing the alcohol-drinking response by administering to a subject an opioid antagonist, wherein the medication is taken one hour before drinking or when the urge to drink alcohol is most compelling would not be most effective and successful in real life.

### SUMMARY OF THE INVENTION

It has been found that the efficacy of extinguishing an alcohol drinking response by administering an opioid antagonist, especially nalmefene, is markedly better within the population of family history positive (FAH+ or FHP) subjects compared to the population of family history negative (FAH− or FHN) subjects. Accordingly, the present invention provides a method of treating alcoholism or alcohol abuse of a FAH+ subject, comprising extinguishing an alcohol-drinking response by administering to the FAH+ subject a pharmaceutically effective amount of an opioid antagonist, especially nalmefene, or a pharmaceutically acceptable salt thereof.

It has now also been discovered that on-demand dosing of an opiate antagonist before imminent alcohol consumption is an effective way of treating alcoholism or alcohol abuse when using the method of extinguishing the alcohol-drinking response. Accordingly, another aspect of the present invention provides a method of treating alcoholism or alcohol abuse, comprising extinguishing an alcohol-drinking response by administering to a subject a pharmaceutically effective amount of an opioid antagonist before imminent drinking. The two above mentioned discoveries can also be employed in combination. Accordingly, the present invention provides a method of treating alcoholism or alcohol abuse of a FAH+ subject, comprising extinguishing an alcohol-drinking response by administering to the FAH+ subject a pharmaceutically effective amount of an opioid antagonist before imminent drinking. Also, the present invention provides a method of treating alcoholism or alcohol abuse of a FAH+ subject, comprising extinguishing an alcohol-drinking response by administering to the FAH+ subject a pharmaceutically effective amount of nalmefene or a pharmaceutically acceptable salt thereof before imminent drinking.

Preferably, the opioid antagonist is selected from the group consisting of naltrexone, naloxone, and nalmefene, more preferably nalmefene. Thus, the present invention provides a method of treating alcoholism or alcohol abuse, comprising extinguishing an alcohol-drinking response by administering to a subject a pharmaceutically effective amount of nalmefene or a pharmaceutically acceptable salt thereof before imminent drinking.

Further, the present invention provides a method of treating alcoholism or alcohol abuse, comprising extinguishing an alcohol-drinking response by administering to a subject transmucosally a pharmaceutically effective amount of an opioid antagonist before imminent drinking. Preferably, the opioid antagonist is administered via oral mucosa. Advantageously, the subject is a FAH+ subject.

Also, the present invention provides a method of treating alcoholism or alcohol abuse, comprising extinguishing an alcohol-drinking response by administering to a subject transmucosally a pharmaceutically effective amount of nalmefene or a pharmaceutically acceptable salt thereof before imminent drinking.

Preferably, the transmucosal preparation useful in the method is one that has rapid onset of action. Accordingly, the present invention provides a method of treating alcoholism or alcohol abuse, comprising extinguishing an alcohol-drinking response by administering to a subject before imminent drinking a transmucosal preparation comprising a pharmaceutically effective amount of an opioid antagonist, wherein the transmucosal preparation has rapid onset of action. Advantageously, the pH of the transmucosal preparation is within the range of about 6 to about 8, preferably about 6.5 to about 7.5.

Additional embodiments and advantages of the invention will be set forth in part in the description that follows, and in part, will be obvious from the description, or may be learned by practice of the invention. The embodiments and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE FIGURES

FIG. 1 describes the maximal administration rate of nalmefene as a function of pH in HCK and MDCK cell membranes.

### DETAILED DESCRIPTION OF THE INVENTION

In one aspect, it has been discovered, that administering an opioid antagonist, especially nalmefene, to a family history positive (FAH+ or FHP) subject suffering from alcoholism or alcohol abuse is surprisingly more effective in a method of treating alcoholism or alcohol abuse by extinguishing an alcohol-drinking response than when nalmefene is administered to a family history negative (FAH− or FHN) subject. In particular, it has been found that when FAH+ subjects are treated by this method, nalmefene decreases drastically the number of heavy drinking days (HDDs) and drinking days (DDs), whereas no clear treatment effect on HDDs or DDs is seen among FAH− subjects. In fact, Example 1 shows that administering orally 40 mg of nalmefene daily to FAH+ subjects reduced the mean number of HDDs about 60% at the fourth month of treatment. Moreover, the ratio of the number of HDDs to DDs within the FAH+ subjects treated daily with 40 mg of nalmefene decreased from 85% to 43%, showing that the treatment changed the pattern of drinking towards moderate drinking.

Further, the method significantly reduces the monthly total alcohol consumption among FAH+ subjects, while hardly no difference is seen among FAH− subjects. For example, the mean number of heavy drinking days during the fourth month of treatment with a daily oral dosage of 40 mg of nalmefene was reduced by 40% (see Example 1).

The term “family history positive subject” or “FAH+ subject” or “FHP subject” as used herein includes subjects who have at least one first degree (father, mother or sibling) or second degree relative (grandparent, uncle or aunt) affected by alcohol problems.

The term “family history negative subject” or “FAH− subject” or “FHN subject” as used herein includes subjects without close relatives with known alcohol problems.

The effective amount of nalmefene, or any other opioid antagonist, or a pharmaceutically acceptable salt thereof is an amount that blocks the stimulatory effects of alcohol.

Nalmefene, or any other opioid antagonist, or a pharmaceutically acceptable salt thereof can be administered daily to the FAH+ subject once per day, or only on those days when drinking occurs. The dose administered will depend upon the age and weight of the patient, and the route of administration, but must be sufficient to assure that the antagonist will be present in sufficient quantities in the body throughout the entire time period of alcohol drinking. Nalmefene, or any other opioid antagonist, can be given by injection, transdermal administration, transmucosal administration, suppository, sublingual administration, oral administration, and the like. A suitable dose for nalmefene using transmucosal delivery is about 1 to about 50 mg, preferably about 5 to about 20 mg. A suitable oral daily dose of nalmefene is about 0.1 to about 300 mg. Suitable dose ranges for natoxone and naltrexone are described below. Suitable dosage forms are described below and, for example, in U.S. Pat. Nos. 5,068,058 and 5,096,715. Nalmefene and other opioid antagonists can also be administered only on demand before imminent drinking as described below.

The subject does not need to commit to abstinence in the beginning of treatment. It is believed that using the methods of the present invention will lead to gradual reduction of alcohol consumption and to less uncontrolled heavy drinking.

The method of the present invention can be used in all FAH+ individuals classified by any of various means as alcoholics or alcohol abusers, except those in which the administration of an opiate antagonist is contraindicated and those suffering from Korsakoffs syndrome. The subject should also undergo medical detoxification if drinking constitutes a hazard that needs to be eliminated immediately, e.g., severe organ damage. The FAH+ status can be determined by screening the family history.

In another aspect, applicants have discovered that administering to a subject suffering from alcoholism or alcohol abuse an effective amount of an opioid antagonist before imminent drinking, i.e., on demand, is sufficient and provides an effective method for reducing alcohol consumption and preventing relapses of heavy drinking. The method comprises extinguishing the alcohol-drinking response by administering to a subject a pharmaceutically effective amount of an opioid antagonist before imminent drinking. The subject suffering from alcoholism or alcohol abuse does not need to plan ahead when drinking alcohol or to evaluate the intensity of alcohol craving before taking an effective dose of an opioid antagonist, but needs to take the medication before imminent drinking. Thus, the method of this aspect of the present invention provides the subject suffering from alcoholism or alcohol abuse a better quality of life by allowing the subject to concentrate on things other than the medication. However, in order for the method of the present invention to be effective, the subject should always have the medication available, and the medication should always be taken before any imminent drinking. Further, the method of the present invention offers better treatment compliance and savings in the cost of treatment.

The method of the present invention also decreases the unwanted effects of the medication. Any opioidergically reinforced behavior, e.g., eating sweets, interest in sex, or jogging, produced while an opioid antagonist is present, will be extinguished because the opioid antagonist blocks the reinforcement. Thus, when the opioid antagonist is taken only before imminent drinking, the subject can better prevent extinction of the above-mentioned competing actions, and enjoy the other behaviors when the opioid antagonist is not present.

The term “imminent drinking” as used herein refers to drinking alcohol that will occur at any moment, for example, within about 1 to about 20 minutes.

In context of the present invention, the term “transmucosally” is intended to include administration via the oral, nasal or lung mucosa.

The term “transmucosal” as used herein refers to pharmaceutical preparations that deliver the pharmaceutically active agent via the oral, nasal or lung mucosa.

In the method of the present invention, the subject does not need to commit to abstinence in the beginning of treatment. It is believed that using the methods of the present invention will lead to gradual reduction of alcohol consumption and to less uncontrolled heavy drinking.

The method of the present invention can be used in all individuals classified by any of various means as alcoholics or alcohol abusers, except those in which the administration of an opiate antagonist is contraindicated and those suffering from Korsakoffs syndrome. The subject should also undergo medical detoxification if drinking constitutes a hazard that needs to be eliminated immediately, e.g., severe organ damage.

Examples of suitable opiate antagonists include but are not limited to nalmefene, naloxone, naltrexone, cyclazocine, diprenorphine, etazocine, levalorphan, metazocine, nalorphine, and their pharmaceutically acceptable salts. Preferred opiate antagonists in the present invention are nalmefene, naloxone and naltrexone, more preferably nalmefene. Naloxone and naltrexone have been approved for use in humans in large scale. Nalmefene injection formulation has been approved for use in humans for reversal of postoperative opiate depression and for management of opiate agonist overdose or toxicity in the U.S.A.

The dose administered will depend upon the age and weight of the patient, the specific opioid antagonist, and the route of administration, but must be sufficient to assure that the antagonist will be present in sufficient quantities in the body throughout the entire time period of alcohol drinking. However, the patient should be free of pharmacologically-active quantities of the antagonist during the days when no drinking is involved. The route of administration should be such that the opioid antagonist is absorbed almost as fast as if injected. However, an injectable preparation is not practical in the method of the present invention since the subject should be able to easily carry and dose the medication. Preferably, the route of administration is transmucosal. The transmucosal route of administration avoids the hepatic first-pass metabolism and possible hepatotoxicity. Preferably, the opioid antagonist is administered via oral mucosa.

The transmucosal preparation can be in various forms, such as in the form of a tablet, a pill, a disc, a patch, a gel or a spray. Preferably, the transmucosal preparation has rapid onset of action allowing rapid blood levels of the opioid antagonist. Suitable transmucosal preparations can be prepared by methods described in the art, such as in U.S. Pat. Nos. 6,248,363; 6,200,604; 6,177,096; 6,159,498; 6,103,266; 5,948,430; or 5,800,832.

For example, a suitable preparation for use in the method of the present invention comprises a film disc having an adhesive layer and a non-adhesive backing layer which are both water soluble and made of pharmacologically-approved materials. The opioid antagonist may be included in either layer, although preferably, it is included in the backing layer which dissolves first. The adhesive layer may comprise at least one film-forming water-soluble polymer, usually a cellulose derivative (the “film-forming polymer”) and at least one pharmacologically acceptable polymer known for its bioadhesive capabilities (the “bioadhesive polymer”). The film forming polymer may comprise hydroxyethyl cellulose, hydroxypropyl cellulose, hydroxypropylmethyl cellulose, hydroxyethylmethyl cellulose, or a combination thereof. The film-forming polymer may be crosslinked or plasticized. The bioadhesive polymer of the adhesive layer may comprise polyacrylic acid (PAA), which may or may not be partially crosslinked, sodium carboxymethyl cellulose (NaCMC), and polyvinylpyrrolidone (PVP), or combinations thereof. The ratio of the bioadhesive polymer to the film-forming polymer in the adhesive layer may vary, depending on the type of pharmaceutical and the amount of pharmaceutical to be used. However, the content of combined components in the adhesive layer can be between 5 and 95% by weight. The non adhesive backing layer may comprise a water-soluble, film-forming pharmaceutically acceptable polymer such as hydroxyethyl cellulose, hydroxypropyl cellulose, hydroxypropylmethyl cellulose, hydroxyethylmethyl cellulose, polyvinylalcohol, polyethylene glycol, polyethylene oxide, ethylene oxide-propylene oxide co-polymers, or a combination thereof. The backing layer component may or may not be crosslinked. The backing layer component may include hydroxyethyl cellulose or hydroxypropyl cellulose. Combinations of different polymers or similar polymers with definite molecular weight characteristics may be used in order to achieve preferred film forming capabilities, mechanical properties, and kinetics of dissolution. For example, the backing layer may contain ¾ of hydroxyethyl cellulose and ¼ of hydroxypropyl cellulose; ⅘ of low molecular weight hydroxyethyl cellulose and ⅕ of medium molecular weight hydroxyethyl cellulose; and 8/9 of low molecular weight hydroxyethyl cellulose and 1/9 of high molecular weight hydroxyethyl cellulose. Crosslinking agents may be used in the backing layer. Suitable crosslinking agents are known in the art, e.g., glyoxal, propylene glycol, glycerol, dihydroxy-polyethylene glycol, and butylene glycol. The amount of crosslinking agent used may vary, depending on the particular polymers and crosslinking agent, but should not exceed 5% molar equivalent of the polymeric material, and preferably comprises 0 to 3% molar equivalent of the polymeric material. The amount of the opioid antagonist to be used depends on the desired treatment strength, and can be from 0.001 to 30% by weight of the preparation. The thickness of the preparation may vary, depending on the thickness of each of the layers. The bilayer thickness can be from 0.05 mm to 1 mm, and more preferably from 0.1 to 0.5 mm. The thickness of each layer may vary from 10 to 90% of the overall thickness of the bilayer preparation. Thus, the preferred thickness of each layer may vary from 0.01 mm to 0.9 mm, and more preferably from 0.03 to 0.6 mm.

The transmucosal preparations described above can be prepared by numerous methods known in the art. For example the components are dissolved in the appropriate solvent or combination of solvents to prepare a solution. Suitable solvents are water, methanol, ethanol, isopropyl alcohol, acetone, methyl ethyl ketone, heptane, or dichloroethane, alone or combination. The final solvent content or residual solvent content in the film may be the result of either or both layers. The solvent may also be used as a plasticizer or dissolution-rate-modifying agent. Each solution is then coated onto a substrate. Each solution is casted and processed into a thin film by techniques known in the art, such as by film dipping, film coating, film casting, spin coating, or spray drying using the appropriate substrate. The thin film is then dried. The drying step can be accomplished in any type of oven. The film layers may be filmed independently and then laminated together or may be filmed one on the top of the other. The film obtained after the two layers have been laminated together or coated on top of each other may be cut into any type of shape, for application to the mucosal tissue. Some shapes include disks, ellipses, squares, rectangles, and parallepipedes.

Especially suitable transmucosal preparations are those prepared by Atrix Laboratories using the BEMA™ (Bioerodible MucoAdhesive) technology. This technology is based on a thin bilayer, Bioerodible MucoAdhesive film, that can provide rapid transmucosal delivery of drug to the systemic circulation. The preparation can be in the form of a small disc that is made up of a backing layer and a mucoadhesive layer containing the opioid antagonist. The disc adheres firmly to the oral mucosa, and solubilization and erosion of the backing layer begins with the opioid antagonist delivered to the mucosal surface as the thin disc naturally erodes.

Suitable transmucosal preparations for use in the method of the present invention include dosage forms that disintegrate rapidly, i.e., within 60 seconds, preferably within 10 seconds, in the mouth thereby ensuring sufficient time for the opioid antagonist on the oral mucosal tissue to facilitate its absorption. The rapidly disintegrating dosage form can be in the form of, for example, a capsule, a tablet (e.g., prepared by RP Scherer Corporation using Zydis® technology), a high viscous liquid or gel, a muco-adhesive, a chewing gum, etc. Rapidly disintegrating dosage forms can be prepared by methods described in the art, for example, in U.S. Pat. Nos. 4,305,502; 4,470,202; 4,754,597; 4,758,598; 5,188,825; 5,343,672; 5,558,880; 5,631,023; 5,648,093; 5,729,958; 5,738,875; or 5,827,541.

For example, suitable rapidly disintegrating dosage forms can be prepared by freezing a composition comprising a solution in a first solvent of a water-soluble or water-dispersible carrier material that is inert towards an opioid antagonist, subliming the first solvent from the frozen composition so as to produce a product having a network of carrier material, adding to the product a solution or suspension of a second non-aqueous solvent containing the predetermined amount of the opioid antagonist, and allowing or causing the second solvent to evaporate. The composition can be frozen in a mold corresponding to the size and shape of the desired shaped dosage form and the first solvent is sublimed, for example in a freeze drier, while the frozen composition is still in the mold. The predetermined amount of the opioid antagonist can then be dosed on to the resulting sublimed product while it is in the mold, or alternatively, the sublimed product can be removed from the mold before the opioid antagonist is dosed on to it. The mould can be, for example, a depression in a metal plate (e.g. an aluminium plate). The plate may contain more than one depression, each depression being of the size and shape corresponding to the desired size and shape of the dosage form. However, the mold is preferably a depression in a sheet of filmic material. The filmic material may contain more than one depression, and it may be similar to that employed in conventional blister packs that are used for packaging oral contraceptive tablets and like medicament forms. For example, the filmic material can be made of thermoplastic material with the depressions formed by thermoforming. Laminates of filmic material such as polyvinyl chloride/polyvinylidene chloride, polyvinyl chloride/polytetrafluorethylene or polyvinyl chloride/polyvinylidene chloride/polyethylene may also be used. If the opioid antagonist is dosed on to the sublimed products while the latter are still in depressions in a sheet of filmic material, a covering sheet may be adhered to the filmic material so as to produce a package enclosing the shaped dosage forms. The covering sheet is preferably an aluminium foil or aluminium foil laminate which may be adhered to the filmic material around the depressions by, for example, a heat sensitive adhesive. The covering sheet is preferably adhered to the filmic material such that it may be peeled away from the filmic material by the user so as to expose the dosage forms in their depressions.

Alternatively, the frozen and sublimed product prior to dosing with the opioid antagonist can be of a size corresponding to the desired size of two or more dosage forms. For example, the composition may be frozen in a tray and the solvent sublimed from the frozen composition to produce a slab or a sheet of sublimed product corresponding in size to that of a number of the desired shaped dosage forms. The sheet may be subdivided to form products of the desired size and the opioid antagonist in the second solvent dosed on to the subdivided products. The subdivision of the sheet does not need to be carried out accurately since the measured amount of the opioid antagonist is added to the subdivided products. If the second solvent does not diffuse excessively through the sheet of sublimed product, the sheet can be dosed with the predetermined amount of opioid antagonist at selected positions on the sheet prior to subdivision and the sheet subsequently subdivided to give shaped dosage forms each containing the predetermined amount of the opioid antagonist.

Advantageously, the pH of the transmucosal preparation is within the range of about 6 to about 8, preferably about 6.5 to about 7.5.

A suitable dose for nalmefene using transmucosal delivery is about 1 to about 50 mg, preferably about 5 to about 20 mg. A suitable dose range for naloxone using transmucosal delivery is about 0.2 to about 40 mg. A suitable dose range for naltrexone using transmucosal delivery is about 1 to about 100 mg. A suitable dosing regime is placing a rapidly disintegrating opioid antagonist preparation upon mucosal surface, such as in the mouth, 1-20 minutes, for example 1-10 minutes, prior to alcohol consumption.

Preferably the opioid antagonist used in the method of the present invention is nalmefene. Nalmefene is a highly specific opiate antagonist without intrinsic agonistic activity.

The invention is also directed to a method of treating alcoholism or alcohol abuse of a FAH+ subject, comprising extinguishing an alcohol-drinking response by administering to the FAH+ subject a pharmaceutically effective amount of an opioid antagonist before imminent drinking. This method can be employed following the teaching of the description.

Further, the present invention is directed to a method of treating alcoholism or alcohol abuse of a FAH+ subject, comprising extinguishing an alcohol-drinking response by administering to the FAH+ subject a pharmaceutically effective amount of nalmefene or a pharmaceutically acceptable salt thereof before imminent drinking. This method can be employed following the teaching of the description.

Opioid antagonists are available on the market or can be prepared by methods known in the art.

The methods of the present invention can be used in connection with other forms of therapies for treating alcoholics, such as punishment of alcohol drinking, procedures to improve will power and social rehabilitation, and coping therapy. Coping therapy is a cognitive therapy on how to cope with small relapses.

The following examples are illustrative, but not limiting, of the methods of the present invention. Other suitable modifications and adaptions of the variety of conditions and parameters normally encountered in clinical therapy and which are obvious to those skilled in the art are within the spirit and scope of the invention.

### EXAMPLE 1

A randomized, double-blind, placebo-controlled, multicenter, parallel group, dose-response study was conducted comparing placebo and two doses of nalmefene. 150 subjects (50 per treatment group) with impaired control of alcohol drinking were enrolled applying the following inclusion criteria: the subject expressed a persistent desire to cut down or control drinking; the subject had difficulty in controlling drinking: the subject often consumed alcohol in larger amounts or for a longer period than was intended; had at least 8 heavy drinking days (5 or more drinks for male, 4 or more drinks for female) within the last month prior to screening; written informed consent obtained; the subject was sober at the time of inclusion; the subject was at the age 18 years or older; and the subject had an address and telephone number, where the subject could be reached.

The subjects were randomly allocated to receive placebo, 10 mg of nalmefene or 40 mg of nalmefene once daily for 16 weeks. Each of the treatment groups had a subgroup having similar numbers of family history positive (FAH+ or FHP) and family history negative (FAH− or FUN) subjects. During their participation in the study, the subjects were allowed to receive general counseling, but not any psychotherapy or other specific alcoholism treatment, including pharmacotherapy. The study consisted of a screening period (screening and inclusion visits) not exceeding 2 weeks, during which the eligibility of the study subjects were evaluated, and follow-up visits at 1, 3, 6, 9, 12, and 16 weeks after the study medication was started.

The alcohol consumption was assessed with the Time Line Follow Back Method. The primary efficacy variable was the number of Heavy Drinking Days (HDDs) per month (defined as 28 days). A Heavy Drinking Day (HDD) was defined as a day when a male subject consumed 5 or more, or a female subject consumed 4 or more standard drinks. A standard drink contains approximately 12 g of ethanol. Secondary efficacy measures included other drinking variables (number of Drinking Days (DDs), ratio of HDDs to DDs, total consumption, and maximum and average intensity of drinking), Obsessive Compulsive Drinking Scale (OCDS), Alcohol Dependence Scale (ADS) and Drinkers Inventory of Consequences -scale (DrInC), clinical laboratory markers (ALAT, GGT, CDT, and MCV) and collateral reports.

The subjects safety was evaluated with monitoring of adverse events with regular blood pressure and heart rate measurements on every visit, and with repeated laboratory tests at weeks 6 and 16.

The monthly drinking of the subjects during the 12 weeks prior to the study was determined first to get a baseline. The total alcohol consumption was 25% higher in the FAH+ subject than in the FAH− subject. The number of HDDs and the number of DDs were at a similar level in all subgroups. The impression of subject's spouse or significant other on how often the subject had appeared clearly intoxicated was estimated by the following question: “Over the past 3 months how would you best describe the frequency of your significant other being intoxicated (noticeably drunk (please choose one and only one answer)?”. The answers did not differ between the FAH+ and FAH− subgroups the majority of the answers being in the categories “several times a a month” and “at least once a week”. The results are shown below in Table 1 and 2.

TABLE 1
Spouse or significant other estimate on the frequency of intoxication
within FAH+ subjects.

| | | Once or | Several times a | At least once | |
|---|---|---|---|---|---|
| | Never | twice | month | a week | Every day |
| Placebo | 2 | 2 | 11 | 13 | 0 |
| | 7% | 7% | 39% | 46% | 0% |
| 10 mg | 1 | 3 | 11 | 12 | 1 |
| | 4% | 11% | 39% | 43% | 4% |
| 40 mg | 1 | 0 | 14 | 7 | 3 |
| | 4% | 0% | 56% | 28% | 12% |

TABLE 2
Spouse or significant other estimate on the frequency of intoxication
within FAH− subjects.

| | | Once or | Several times a | At least once | |
|---|---|---|---|---|---|
| | Never | twice | month | a week | Every day |
| Placebo | 0 | 2 | 4 | 10 | 0 |
| | | 12% | 25% | 63% | 0% |
| 10 mg | 0 | 3 | 4 | 6 | 2 |
| | | 20% | 27% | 40% | 13% |
| 40 mg | 1 | 2 | 5 | 6 | 1 |
| | 7% | 13% | 33% | 40% | 7% |

The results of the study showed that within the FAH+ subjects receiving nalmenfene 40 mg, the mean number HDDs was markedly lower during each treatment month than at baseline (change from 14.5 to 6.0). During the 4^{th} month, the mean number of HDDs was about 60% smaller than at baseline. In the 10 mg group, there was a smaller reduction in the early phase of the treatment within the FAH+ subjects. Towards the end of the study, the number of HDDs tended to return towards the baseline values. In the placebo group, the mean number of HDDs gradually declined from 14.4 to 11.0 within the FHP positive subjects. Repeated measures ANOVA revealed a significant treatment by month interaction. Within the FAH− subjects, no clear treatment effects were seen in the number of HDDs. Repeated measures ANOVA revealed a significant effect of month but no significant treatment by month interaction. Further, a clear reduction in the number of DDs was observed within the FAH+ subjects receiving nalmefene 40 mg.

As a descriptive indicator of the drinking pattern, the ratio of the number of HDDs to DDs was calculated. In the FAH+ subjects receiving 40 mg nalmefene, the ratio decreased from 85% down to 43% indicating, that the pattern of drinking changed towards moderate drinking. The changes were less remarkable in other groups.

Further, the results showed that the monthly total alcohol consumption (number of standard drinks) decreased in all treatment groups within the FAH+ subjects. The decreases were most pronounced in the group receiving 40 mg of nalmefene where the mean number of drinks during the 4^{th} month was only about 40% of the number of drinks at the baseline. Repeated measures ANOVA revealed a significant effect of month and a non-significant treatment by month interaction. Within the FAH− subjects, the mean number of drinks remained almost unchanged throughout the study.

The estimate on the subject's frequency of intoxication by subject's spouse or significant other was obtained again at the end of the study. Within the FAH+ subjects, there were more subjects in nalmefene 40 mg groups in categories “never” and “once or twice” than in other groups, and no subjects in the categories “at least once a week” or “every day”. This result was statistically significant (treatment; Mantel-Haenszel Chi-Square test for linear association, exact test). The results are shown in Table 3 and 4 below.

TABLE 3
Spouse or significant other estimate on the frequency of intoxication
within FAH+ subjects at week 16.

| | | Once or | Several times a | At least once | |
|---|---|---|---|---|---|
| | Never | twice | month | a week | Every day |
| Placebo | 2 | 6 | 9 | 7 | 0 |
| | 8% | 25% | 38% | 29% | 0% |
| 10 mg | 1 | 6 | 8 | 4 | 0 |
| | 5% | 32% | 42% | 21% | 0% |
| 40 mg | 3 | 9 | 14 | 0 | 0 |
| | 14% | 41% | 45% | 0% | 0% |

TABLE 4
Spouse or significant other estimate on the frequency of intoxication
within FAH− subjects at week 16.

| | | Once or | Several times a | At least once | |
|---|---|---|---|---|---|
| | Never | twice | month | a week | Every day |
| Placebo | 0 | 4 | 2 | 4 | 0 |
| | | 40% | 20% | 40% | 0% |
| 10 mg | 1 | 4 | 4 | 1 | 1 |
| | 9% | 36% | 36% | 9% | 9% |
| 40 mg | 2 | 3 | 5 | 1 | 1 |
| | 15% | 23% | 38% | 8% | 8% |

The study shows that the efficacy of 40 mg nalmefene once daily in reducing alcohol drinking in FAH+ subjects appeared consistent across all variables examined, as the results on serum GGT and significant other estimated were in concordance with drinking data. There were marked reductions both in the number of drinking occasions (drinking days) and in the amount of alcohol consumed per occasion (ratio of HDDs to DDs). The latter points to an enhanced level of control over drinking. Also the FAH+ subjects receiving 10 mg nalmefene appeared to have a positive tendency in the total alcohol consumption and in the number of HDDs during the first month of treatment. A substantial effect of nalmefene in FAH+ subjects was noted without an intensive structured psychosocial therapy. In FAH− subjects, there was a trend toward decreased alcohol consumption early in the treatment with nalmefene. Nevertheless, no dose response could be detected and the treatment effects were not distinguishable from placebo.

### EXAMPLE 2

The diffusion relations in different biological membranes are known and, thus, transmucosal behaviour of a drug can be reliably estimated (Rojanasakul, Y, et al., Pharm. Res. 9:1029-1034 (1992)). In the oral mucosa, the buccal membrane corresponds to the eye cornea with regard to permeability, and the sublingual membrane corresponds to MDCK cell (Madin-Darby canine kidney) membrane. In vitro membrane diffusion studies on nalmefene were conducted using eye cornea cell (HCE) and MDCK cell membranes.

Preparation of HCE cell culture. The cell culture was prepared as described by Toropainen et al. (JOVS 42:2942-2948 (2001)). Accordingly, polyester filters (surface area 4.7 cm², pore size 0.4 μm, Transwell Clear, Costar, Cambridge, Mass.) were coated with 275 μl of rat tail collagen type I (1.3 mg/ml; Becton Dickinson, Bedford, Mass.). Collagen was allowed to gel on the filters at room temperature for at least 4 hours after which immortalized human cornea epithelial cells (HCE cells) seeded onto the coated filters (passage 33, about 90,000 cells/cm²). The cells were grown on the filters in a culture medium both in apical and basolateral chambers for a week. Within this time the cells were confluent. The cells were then exposed to an air-liquid interface (the apical chamber did not have the culture medium) for two weeks. During the whole culture period, the cells were grown in an incubator at 37° C. in humidified air with 5% CO₂. The culture medium was replaced every other day. TER (Transepithelial Electrical Resistance; Endohm, World Precision Instruments, Sarasota, Fla.) was used as an indicator of epithelial differentiation and epithelial tightness. The culture medium (SHEM medium) consisted of DMEM/F12 (Dulbecco's MEM/nutrient Mix F 12 (1:1); Gibco, Paisley, Scotland), which included 0.3 mg/ml L-glutamine (Gibco, Paisley, Scotland), 15% (v/v) heat-inactivated fetal bovine serum (FBS, Gibco, Paisley, Scotland), 500 IU/ml penisillin and 5000 μg/ml streptomycin (both from Gibco, Paisley, Scotland), 10 ng/ml epidermal growth factor (EGF, Calbiochem, La Jolla, Calif.), 5 μg/ml insulin (Sigma Chemicals Co, St. Louis, USA), 0.5% (v/v) dimethyl sulfoxide (DMSO; Sigma, St. Louis, Mo.) and 0.1 μg/ml cholera toxin (Calbiochem, La Jolla, Calif.).

Preparation of MDCK cell culture: Polycarbonate filters (surface area 4.7 cm², pore size 0.4 μm; Transwell, Costar, Cambridge, Mass.) were coated the same way as the polyester filters above. MDCK cells were seeded onto the coated filters at a concentration of about 100,000 cells/cm². The culture medium was both in apical and basolateral chambers during the whole culture period, and the culture medium was changed every day. The cells were confluent within three days. The culture medium consisted of DMEM/F12 (Dulbecco's MEM/nutrient Mix F 12 (1:1), Gibco, Paisley, Scotland), which included 0.3 mg/ml L-glutamine (Gibco, Paisley, Scotland), 10% (v/v) heat-inactivated fetal bovine serum (FBS, Gibco, Paisley, Scotland), 500 IU/ml penisillin and 5000 μg/ml streptomycin (both from Gibco, Paisley, Scotland). The incubator environment was the same as described above.

In vitro permeation study: When the TER of the filter cultured HCE cells was 400-500 Ω×cm², the cells were used in the nalmefene permeation study. Before the study, the apical and basolateral sides were rinsed with a BSS Plus buffer solution (BSS, Alcon, Fort Worth, Tex.), containing 10 mM of HEPES (Gibco, Paisley, Scotland). The penetration study was initiated by adding 2.6 ml of BSS Plus/10 mM HEPES solution to the basolateral side and 1.5 ml of nalmefene solution (189 μg/ml; 500 μM) to the apical side. The nalmefene solutions were prepared in four different pH levels: pH 6.5, pH 7.0, pH 7.5, and pH 8.0. At 15, 30, 45, 60, 75, 90, 105, 120, 150, and 180 minutes, aliquots of 400 μl were withdrawn from the basolateral chamber and replaced with an equal volume of blank buffer solution.

MDCK cells were used for the permeation study on the 4^{th} day when the TER value was about 250 Ω×cm². HBSS (Gibco, Paisley, Skotlanti) /10 mM HEPES was used a buffer solution. The permeation study was performed as described above. All the permeation studies were performed at 37° C. using a horizontal plate mixer (Heidolph Inkubator 1000 and Titramax 1000; Heidolph Electro GmbH & Co., Kelheim, Germany). After the studies, TER was metered. The TER data were not changed during the permeability studies.

The nalmefene solutions of each pH were tested using four membranes. The permeability coefficients were calculated for each membrane including the mean and variation. The apparent permeability coefficients were calculated as:

Pₐₚₚ=J/(C×S), (Equation 1)

where Pₐₚₚ is the apparent permeability coefficient, J is the drug flux across the membrane (μg/min), C is the concentration of nalmefene on the apical side (donor side) (μg/ml) and S is the surface are of the filter (4.2 cm²).

Maximal drug flux (Jₘₐₓ: The drug flux can be calculated using Equation 1 as follows:

J=Pₐₚₚ×C×S (Equation 2)

Thus, the maximal flux Jₘₐₓ is calculated as Jₘₐₓ=Pₐₚₚ×Cₛ per cm². Cₛ is the water solubility of nalmefene. The water solubility of nalmefene as a function of pH is described in Table 5 (Merck-Index, 19^{th} Ed., 2001):

TABLE 5
Solubility of Nalmefene HCl in Water as a Function of pH

| pH | Solubility in water (mg/ml) |
|---|---|
| 2.25 | 128 |
| 5.71 | 131 |
| 6.115 | 133 |
| 6.25 | 124 |
| 7.85 | 1.09 |
| 8.5 | 0.180 |
| 9.15 | 0.090 |
| 10.4 | 0.230 |

Because the water solubility of nalmefene was not available in the literature for all the pH values used in the permeation study, the solubilities at pH values 6.5, 7.0, 7.5, and 8.0 were determined matematically from a diagram that described the logarithm of the solubility listed on Table 5 as a function of pH (C_{s,pH}). The results are shown in Table 6.

TABLE 6
Matematically Determined Solubility of Nalmefene HCl in Water as a
Function of pH

| pH | Solubility in water (mg/ml) |
|---|---|
| 6.5 | 111.9 |
| 7.0 | 88.6 |
| 7.5 | 31.9 |
| 8.0 | 0.44 |

The maximal flux of nalmefene at each pH through 10 cm² membrane was calculated using Equation 3:

J_{max,10 cm2}=Pₐₚₚ×C_{s,pH}×10 cm² (Equation 3)

Results

1. Liposolubility and water solubility

Log P (octanol/water) value was calculated for nalmefene, 2.66, using KowWin program (http://esc.syrres.com/interkow). This value represents nalmefene's liposolubility in the non-ionized form. The result shows that nalmefene is moderately lipophilic. The pKₐ for nalmefene is 7.63 and its water solubility (Cₛ) is dependent on pH (see Table 5).

2. Permeability

The permeability of nalmefene in MDCK and HCE membranes is dependent on pH. The permeability factors for nalmefene in HCE and MDCK membranes are presented in Table 7.

TABLE 7
Permeability Factors (Pₐₚₚ) for Nalmefene in HCE and MDCK Cell
Membranes (n = 4)

| | HCE | MDCK |
|---|---|---|
| pH | (Pₐₚₚ ± SD) × 10⁻⁶ cm/s | (Pₐₚₚ ± SD) × 10⁻⁶ cm/s |
| 6.5 | 7.86 ± 0.29 | 11.38 ± 0.59 |
| 7.0 | 12.11 ± 0.79 | 18.27 ± 0.16 |
| 7.5 | 13.28 ± 0.61 | 22.71 ± 1.08 |
| 8.0 | 13.55 ± 1.64 | 23.87 ± 2.49 |

Since there were no changes in the TER values of the cells during the study, the changes in the permeability constants are due to the changing liposolubility of nalmefene at different pH values.

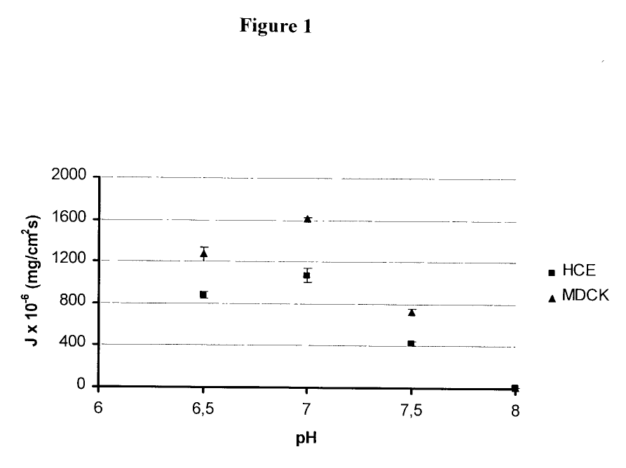

Maximal administration rate of nalmefene: The maximal flux of nalmefene in HCE and MDCK cell membranes is strongly dependent on pH. As shown by Equation 3, the maximal flux is dependent on both the permeability and the water solubility at the pH value in question. Combining the permeability and water solubility factors, the maximal administration rate of nalmefene (mg/cm²s) was estimated as a function of pH in HCE and MDCK cell membranes. The results are shown in Table 8 and FIG. 1.

TABLE 8
Maximal Administration Rate (Jₘₐₓ × 10⁻⁶) for Nalmefene in HCE
and MDCK Cell Membranes (n = 4)

| | HCE | MDCK |
|---|---|---|
| | Jₘₐₓ × 10⁻⁶ ± SD | Jₘₐₓ × 10⁻⁶ ± SD |
| pH | mg/(cm² s) | mg/(cm² s) |
| 6.5 | 879 ± 32 | 1273 ± 66 |
| 7.0 | 1072 ± 70 | 1618 ± 14 |
| 7.5 | 423 ± 19 | 723 ± 34 |
| 8.0 | 6.0 ± 0.7 | 10.6 ± 1.1 |

Pharmacokinetic simulation: A pharmacokinetic simulation model was applied to nalmefene using STELLA® software (High Performance Systems, Inc.) to estimate the concentration of nalmefene in plasma after intraoral administration using the previously calculated J_{max,10 cm2} as a flux of the drug into the plasma. This model was based on the kinetics of nalmefene after i.v. administration described by Dixon el al.(Clin. Pharmacol Ther. 39:49-53 (1986)). Dixon el al shows that nalmefene has a 3-phase kinetics. The simulation model was tested by comparing the results from the simulation to the actual concentrations in plasma after i.v. administration. The results of the simulation show that it is possible to achieve similar maximal concentrations in plasma after administration of nalmefene through oral mucosa as those achieved by oral administration within a relatively short period of time, e.g., after a 10 minute contact of nalmefene with the oral mucosa.

Conclusions: It was found that the local pH in the nalmefene molecule micro environment is an essential factor in designing suitable pharmaceutical formulations for intraoral administration. According the results of permeation study, the pH of the oral transmucosal formulation containing nalmefene should be more than 6 and less than 8, preferably in the pH range of 6.5-7.5. Such pH conditions can be achieved, e.g., by using suitable additives, such as for example organic and/or inorganic salts and buffers, in the formulation in question. In addition to the pH, the contact area and the contact time of the dosage form should be defined for each formulation type in order to finalize the desired nalmefene administration properties.

Those skilled in the art will recognize that while specific embodiments have been illustrated and described, various modifications and changes may be made without departing from the spirit and scope of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. All publications, patent applications and patents cited herein are fully incorporated by reference.

What is claimed is:

1. A method of treating alcoholism or alcohol abuse, comprising extinguishing an alcohol-drinking response by administering to a subject a pharmaceutically effective amount of an opioid antagonist before imminent drinking.

2. The method according to claim 1, wherein the opioid antagonist is selected from the group consisting of nalmefene, naloxone, naltrexone, cyclazocine, diprenorphine, etazocine, levalorphan, metazocine, and nalorphine, or a pharmaceutically acceptable salt thereof.

3. The method according to claim 2, wherein the opioid antagonist is selected from the group consisting of nalmefene, naloxone and naltrexone, or a pharmaceutically acceptable salt thereof.

4. The method according to claim 3, wherein the opioid antagonist is nalmefene or a pharmaceutically acceptable salt thereof.

5. A method of treating alcoholism or alcohol abuse, comprising extinguishing an alcohol-drinking response by administering to a subject transmucosally a pharmaceutically effective amount of an opioid antagonist before imminent drinking.

6. The method according to claim 5, wherein the opioid antagonist is selected from the group consisting of nalmefene, naloxone, naltrexone, cyclazocine, diprenorphine, etazocine, levalorphan, metazocine, and nalorphine, or a pharmaceutically acceptable salt thereof.

7. The method according to claim 6, wherein the opioid antagonist is selected from the group consisting of nalmefene, naloxone and naltrexone, or a pharmaceutically acceptable salt thereof.

8. The method according to claim 7, wherein the opioid antagonist is nalmefene or a pharmaceutically acceptable salt thereof.

9. A method of treating alcoholism or alcohol abuse, comprising extinguishing an alcohol-drinking response by administering to a subject before imminent drinking a transmucosal preparation comprising a pharmaceutically effective amount of an opioid antagonist, wherein the transmucosal preparation has rapid onset of action.

10. The method according to claim 9, wherein the opioid antagonist is selected from the group consisting of nalmefene, naloxone, naltrexone, cyclazocine, diprenorphine, etazocine, levalorphan, metazocine, and nalorphine, or a pharmaceutically acceptable salt thereof.

11. The method according to claim 10, wherein the opioid antagonist is selected from the group consisting of nalmefene, naloxone and naltrexone, or a pharmaceutically acceptable salt thereof.

12. The method according to claim 11, wherein the opioid antagonist is nalmefene or a pharmaceutically acceptable salt thereof.

13. The method according to claim 8 or 12, wherein the dose is about 1 to about 50 mg.

14. The method according to claim 13, wherein the dose is about 5 to about 20 mg.

15. The method according to claim 9, wherein the transmucosal preparation is administered orally.

16. The method according to claim 15, wherein the transmucosal preparation comprises a backing layer and a mucoadhesive layer, and wherein the mucoadhesive layer comprises an opioid antagonist.

17. The method according to claim 15, wherein the transmucosal preparation is a rapidly disintegrating capsule or tablet.

18. The method according to claim 15, wherein the pH of the transmucosal preparation is within the range of about 6 to about 8.

19. The method according to claim 18, wherein the pH of the transmucosal preparation is within the range of about 6.5 to about 7.5.

20. The method of claim 1 or 5, wherein said method is repeated prior to each episode of alcohol consumption by the subject.

21. The method according to any one of claims 1, 5, or 9, wherein the subject is a FAH+ subject.

22. A method of treating alcoholism or alcohol abuse of a FAH+ subject, comprising extinguishing an alcohol-drinking response by administering to the FAH+ subject a pharmaceutically effective amount of an opioid antagonist.

23. The method according to claim 22, wherein a pharmaceutically effective amount of nalmefene or a pharmaceutically acceptable salt thereof is administered.
